(19) United States
(12) Plant Patent Application Publication
(10) Pub. No.: US 2022/0338402 P1
(43) Pub. Date: Oct. 20, 2022

(54) Phlox plant named 'Eye Caramba'

(50) ;

(71) Applicant: Hans A. Hansen, Zeeland, MI (US)

(72) Inventor: Hans A. Hansen, Zeeland, MI (US)

(73) Assignee: Walters Gardens Inc, Zeeland, MI (US)

(21) Appl. No.: 17/300,221

(22) Filed: Apr. 16, 2021

(51) Int. Cl. A01H 6/70

(52) U.S. Cl. PLT/320

(57) ABSTRACT

A unique cultivar of Creeping Phlox plant named ‘Eye Caramba’ characterized by vigorous, dense, rapidly-spreading, multi-stemmed, winter-hardy habit with short, bright green, narrow leaves. Flowering begins in late April and continuing for up to five weeks in cooler weather conditions, on heavily-branched peduncles and completely cover the plant in peak season. Petals of newly opened flowers are deep purplish-pink with a reddish-pink eye and a small notch in the tips of the petals. The new plant is able to withstand dry conditions once established, and the foliage stays clean and resists mildew. The new plant is especially suitable for the landscape as a potted plant and in the garden as a specimen or en masse.

Botanical classification: Phlox subulata.

Variety denomination: ‘Eye Caramba’.

### STATEMENT REGARDING PRIOR DISCLOSURES UNDER 37 CFR 1.77(b)(6)

The first non-enabling disclosure of the claimed plant, in the form of a photograph and brief description on a website operated by Walters Gardens, Inc. on Feb. 1, 2020. Subsequently, the new plant was advertised in the “Walters Gardens 20-21 Catalog” by Walters Gardens, Inc. released on May 20, 2020. The claimed plant was first sold to the public on Jul. 13, 2020 by Walters Gardens, Inc., who obtained the plant and all information relating thereto, from the inventor. No plants of Phlox subulata ‘Eye Caramba’ have been sold to the public in this country or anywhere in the world, nor has any disclosure of the new plant been made, more than one year prior to the filing date of this application, and such sale or disclosure within one year was either derived directly or indirectly from the inventor.

### BACKGROUND OF THE INVENTION

The present invention relates to a new and distinct cultivar of Creeping Phlox plant known as Phlox ‘Eye Caramba’ and will be referred to hereafter by its cultivar name, ‘Eye Caramba’, or the “new plant”. The new plant was hybridized by the inventor at a wholesale perennial nursery in Zeeland, Mich. on Apr. 15, 2015 as a cross between Phlox subulata ‘North Hills’ (not patented) as the female or seed parent and Phlox subulata ‘Early Spring Light Pink’ (not patented) as the male or pollen parent. The new plant passed initial evaluation on the spring of 2017 and was assigned the breeder code 15-18-x through the remaining evaluation process. ‘Eye Caramba’ was first asexually propagated by stem cuttings in the greenhouses at the same nursery in Zeeland, Mich. in the summer of 2017. The unique characteristics of the new plant have been found to be reproducible and stable in successive generations of asexually propagated and the resultant plants have been found to be identical to the original selection.

### BRIEF SUMMARY OF THE PLANT

Phlox ‘Eye Caramba’ is unique from all other Creeping Phlox known to the inventor. The nearest comparison plants known to the inventor include: ‘Eye Candy’ U.S. Plant Pat. No. 31,484, ‘Drummond's Pink’ (not patented), ‘McDaniel's Cushion’ (not patented), ‘Emerald Pink’ (not patented), ‘Coral Eye’ (not patented), ‘Eye Shadow’ U.S. Plant Pat. No. 31,169, ‘Crater Lake’ U.S. Plant patent application Ser. No. 16/974,099 and ‘Plumtastic’ U.S. Plant Pat. No. 28,896.

‘Eye Candy’ has flowers that are light lavender-pink with larger notched apices and wine-purple eye. ‘Drummond's Pink’ has similar flower petal color but the eye is less significant. ‘McDaniel's Cushion’ has similar flower petal color but the eye is smaller and darker purple. ‘Emerald Pink’ has a lighter pink flower color and the eye is smaller and the eye is much smaller. ‘Coral Eye’ has a much lighter pink flower that becomes near white, and the large eye is rosy-red. ‘Eye Shadow’ has flowers with rosy purple petals and a dark purple eye. ‘Crater Lake’ has a faster growth rate and earlier flowers that are a lighter cool indigo purple with smaller dark purple eye marks. ‘Plumtastic’ has flowers of violet-pink with purple markings near the eye, and the flower initially has a light, near-white center that darkens to the same violet-pink at maturity.

The female parent, ‘North Hills’, has smaller near white with a blush of light purple flowers with a darker purple eye. The male parent, ‘Early Spring Light Pink’ has flowers that are light pink.

Phlox ‘Eye Caramba’ differs from and all other Phlox known to the inventor in the following repeatedly observed traits in combination:
1. Vigorous plants of dense, rapidly-spreading habit, spreading by rooting stems, producing short, clean, glabrous, bright-green, narrow leaves;
2. Multiple heavily-branched stems produce branched panicles;
3. Flower beginning in late April and continuing for up to five weeks, in cool conditions, completely covering plant at peak flowering;
4. Flowers of deep purplish-pink with a reddish-pink eye and small notches at the tip of the petals;
5. Plant is able to withstand dry conditions once established.

### BRIEF DESCRIPTION OF THE DRAWINGS

The photographs of the new plant demonstrate the unique traits of Phlox ‘Eye Caramba’ and the overall appearance of two-years-old plants growing in a partially shaded greenhouse in Zeeland, Mich. The colors are as accurate as reasonably possible with color reproductions. Variation in ambient light spectrum, source and direction may cause the appearance of minor variation in color.

FIG. 1 shows a landscape habit view of the new plant in early flowering stages with foliage.

FIG. 2 shows a close-up of the flowers and buds.

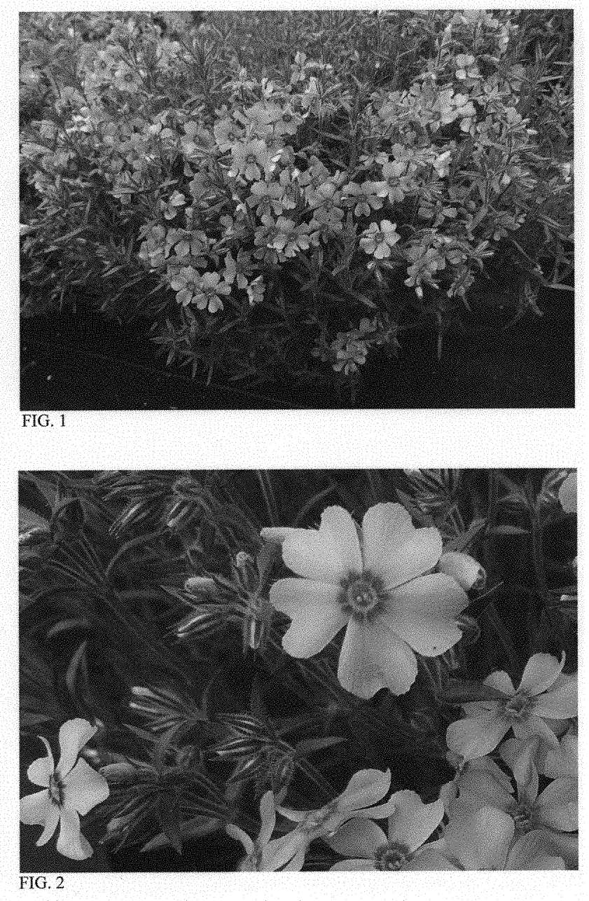

### DETAILED BOTANICAL DESCRIPTION

The following descriptions and color references are based on the 2015 edition of The Royal Horticultural Society Colour Chart except where common dictionary terms are used. Phlox ‘Eye Caramba’ has not been observed under all possible environments. The phenotype may vary slightly with different growing environments such as temperature, light, fertility, soil pH, moisture and maturity levels, but without any change in the genotype. The following observations and size descriptions are based on three-year-old plants in the full-sun trial garden of a wholesale perennial nursery in Zeeland, Mich. with supplemental fertilizer and water as needed.
Botanical classification: Phlox subulata;
Parentage: Female or seed parent and male or pollen parent is Phlox subulata ‘North Hills’ and the and male or pollen parent is ‘Early Spring Light Pink’;
Plant habit: Winter-hardy, evergreen herbaceous perennial; short, dense, producing about 20 to 40 stiff, highly-branched prostrate stems; foliage to 14.0 cm tall and 26.0 cm wide, average 13.5 cm tall and 66.0 cm wide; flowering to 14.5 cm tall and 30.0 cm wide;
Propagation: Stem cuttings; rooting in about 3 weeks;
Time to produce finished crop in 3.8 liter pots: About 8 to 10 weeks; vigorous;
Root: Fibrous and freely branching; color creamy white to tan depending on soil type;
Leaves: Simple; four-ranked; apex apiculate; base truncate, clasping; margin entire and micro-ciliolate; glabrous and matte both adaxial and abaxial; about 15.0 mm long by about 3.8 mm wide just above base;
Leaf color: Adaxial expanding and mature nearest RHS 137C, abaxial both expanding and mature nearest RHS 138A; base adaxial and abaxial nearest RHS 145C;
Foliage fragrance: None detected;
Veins: Pinnate; not conspicuous adaxial and abaxial;
Vein color: Same color as surround leaf;
Petiole: Leaves sessile;
Stems: Cylindrical; flexible; wiry; prostrate; highly branching; to about 15.0 cm long and 2.5 mm diameter near base;
Stem color: Color nearest RHS 195A when exposed;
Nodes: Proximally about 3.0 mm apart; distally less than 1.0 mm apart;
Node color: Color nearest RHS 195A;
Inflorescence: Upright to outwardly; about 6.5 cm long and 4.0 cm wide; up to 3 flowers per branch and 3 branches per stem;
Flowers: Perfect; salverform; mostly flat faced; about 17.0 mm across face and 12.0 mm long; with fused corolla tube about 10.0 mm long and 2.0 mm diameter near face; attitude upright to outwardly;
Flower longevity: About 5 days on plant; self-cleaning;
Flower fragrance: Very faintly sweet;
Buds one to two days prior to opening: Narrowly elliptical; bluntly acute to rounded apex with rounded base; petals implicate; about 10.0 mm long; 2.5 mm diameter in the bulb and 6.5 mm long in tube; corolla tube to 2.0 mm diameter and about 3.5 mm long;
Bud color: Exposed petal bulb portion between RHS 75C and RHS 75B; proximal corolla tube under sepals nearest RHS N144D; calyx nearest RHS N186B where exposed to high light and in lower light exposure nearest RHS N137B;
Petals: Five; obtuse; consisting of limb and basal claw fused into corolla tube; apex rounded and shallowly emarginate, cleft to about 1.0 mm deep; limbs slightly imbricate; margin entire; glabrous adaxial and abaxial;
Petal size: Limb about 7.0 mm long and 7.0 mm wide near apex; tube about 10.0 mm long and 2.0 mm diameter;
Petal color upon opening:
   Adaxial.—Limb between RHS 72D and RHS 73A with eye ring about 1.5 mm wide nearest RHS 61A; proximal 3.0 mm of corolla tube between RHS 145D and RHS 145C, remaining distal tube portion nearest RHS 72D.
   Abaxial.—Limb between RHS 73A and RHS 73B, proximal 1.0 mm of tube nearest RHS 145C, next proximal 2.0 mm nearest RHS NN155A, remaining distal tube with striated portion nearest RHS 70B and RHS 73D.
Petal color upon maturity:
   Adaxial.—Limb between RHS 72D and RHS 73A with two marks near center of about 1.0 mm long and 0.5 mm wide nearest RHS 61A, proximal 3.0 mm of corolla tube between RHS 145D and RHS 145C, remaining distal tube portion nearest RHS 72D.
   Abaxial.—Limb between RHS 73A and RHS 73B lightening near center between RHS 73A and RHS NN155D proximal 1.0 mm of tube nearest RHS 145C, next proximal 2.0 mm nearest RHS NN155A, remaining distal tube with striated portion nearest RHS 70B and RHS 73D.
Androecium: Typically five;
   Filaments.—Typically five, adnate to inner corolla to various heights about 5.0 mm to 8.0 mm from base; free in the distal 1.0 mm to about 2.0 mm long and 0.2 mm in diameter; color nearest RHS NN155D.
   Anther.—Five; oblong elliptic; dorsifixed; about 1.0 mm long by 0.5 mm wide; color nearest RHS 17A.
   Pollen.—Nearly microscopic; color nearest RHS N25C.
Gynoecium: One pistil per flower; 8.0 mm long;
   Style.—Cylindrical; about 6.0 mm long and 0.3 mm diameter when flower is mature; persistent after flower abscission; color nearest RHS 1D.
   Stigma.—Trifid in distal 2.0 mm long, about 0.3 mm diameter; color nearest RHS 1C.
   Ovary.—Inferior; conical; glabrous; acute apex and truncate base; about 1.0 mm long and 1.0 mm diameter; color nearest RHS 143A.
Calyx: Campanulate; pubescent abaxial, glabrous adaxial; about 7.0 mm long and 3.0 mm across at apex;
Sepals; Five; linear; glabrous and lustrous adaxial, matte and pubescent to puberulent abaxial; acute apex; fused in basal 4.0 mm; margin entire; individually about 7.0 mm long and 1.0 mm wide at fusion;
Sepal color: Adaxial nearest RHS 137B, fused margin portions nearest RHS 157D; abaxial nearest RHS 138A in lower light, and in high light exposure of nearest RHS N186C with margins nearest RHS 157D;
Peduncle: Puberulent; strong, flexible; mostly upright; cylindrical; to about 2.0 mm diameter at base and 2.5 cm long;
Peduncle color: Low light or ventrally nearest RHS 147C; high light or dorsally between RHS 182B and RHS N186C;
Pedicle: Cylindrical; glabrous; flexible; upright to outwardly; variable lengths from about 4.0 mm to 8.0 mm long and 0.7 mm diameter;
Pedicle color: Variable depending on light exposure; ventrally nearest RHS 186A, dorsally nearest RHS N186C;
Fruit and seeds: Not observed;
Hardiness and culture: The new plant grows best with full sun, light moisture and deep drainage; hardy to at least from USDA zone 3 through 9.
Disease and pest resistance: Phlox ‘Eye Caramba’ demonstrates excellent powdery mildew resistance under conditions that would normally show symptoms.

I claim:

1. A new and distinct cultivar of Creeping Phlox, Phlox plant named ‘Eye Caramba’, as herein described and illustrated.
